(19) United States
(12) Patent Application Publication
(10) Pub. No.: US 2020/0121739 A1
(43) Pub. Date: Apr. 23, 2020

(54) BACTERIA FOR TREATING CANCER

(71) Applicant: Evelo Biosciences, Inc., Cambridge, MA (US)

(72) Inventors: Brian Goodman, Jamaica Plain, MA (US); Leslie Wardwell-Scott, Brookline, MA (US); Alexandra Sirota-Madi, Brookline, MA (US); Erin B. Troy, Cambridge, MA (US); Samuel W. Andrewes, Somerville, MA (US)

(21) Appl. No.: 16/477,478

(22) PCT Filed: Jan. 18, 2018
(86) PCT No.: PCT/US18/14209

§ 371 Date: Jul. 11, 2019

Related U.S. Application Data
(60) Provisional application No. 62/447,730, filed on Jan. 18, 2017.

(51) Int. Cl. A61K 35/742; A61K 35/744; A61K 45/06; A61K 35/74

(52) U.S. Cl. CPC A61K 35/742; A61K 35/74; A61K 45/06; A61K 35/744

(57) ABSTRACT

Provided herein are methods of treating cancer and/or augmenting a microbiome in a subject who has a tumor.

### RELATED APPLICATION

This application claims the benefit of priority to U.S. Provisional Patent Application Ser. No. 62/447,730, filed Jan. 18, 2017, which is hereby incorporated herein by reference in its entirety.

### SUMMARY

In certain aspects, provided herein are methods and compositions related to the treatment of a cancer in a subject (e.g., a human subject) who has a tumor (e.g., a colon tumor). In some embodiments, the method comprises augmenting a microbiome in the subject (e.g. augmenting the tumor microbiome).

In some embodiments, the method comprises administering to the subject a pharmaceutical composition comprising a bacterium of a genus underrepresented in tumors compared to normal tumor-adjacent tissue (e.g., a bacterium of a genus listed in Table 1) and/or of a species underrepresented in tumors compared to normal tumor-adjacent tissue (e.g., a bacterium of a species listed in Table 2). In some embodiments, the pharmaceutical composition comprises bacteria of two or more genera listed in Table 1 (e.g., at least 3, 4, 5, 6, 7, 8, 9, 10, 11, or 12 of the genera listed in Table 1) and/or two or more species listed in Table 2 (e.g., at least 3, 4, 5, 6, 7, 8, 9, 10, 11, or 12 of the species listed in Table 2). In some embodiments, at least 60% (e.g., at least 65%, at least 70%, at least 75%, at least 80%, at least 85%, at least 90%, at least 95%, at least 98%, at least 99% or essentially 100%) of the bacteria in the pharmaceutical composition are bacteria of a genus listed in Table 1 or of a species listed in Table 2. In some embodiments, the pharmaceutical composition comprises at least 1×10⁶ colony forming units (CFUs) (e.g., at least 2×10⁶ CFUs, at least 5×10⁶ CFUs, at least 1×10⁷ CFUs, at least 5×10⁷ CFUs, at least 1×10⁸ CFUs, at least 5×10⁸ CFUs, at least 1×10⁹ CFUs) of bacteria in the pharmaceutical composition are bacteria of a genus listed in Table 1 or of a species listed in Table 2. In some embodiments, the pharmaceutical composition is administered in multiple doses (e.g., 2, 3, 4 or 5 doses). In some embodiments, an antibiotic is administered to the subject before administration of the pharmaceutical composition. In some embodiments, the pharmaceutical composition is administered by intratumoral, subtumoral, and/or peritumoral injection. In some embodiments, administration of the pharmaceutical composition induces tumor cell death. In some embodiments, administration of the pharmaceutical composition induces an anti-tumor immune response.

In some embodiments, the method comprises administering to the subject a pharmaceutical composition that depletes the tumor of a bacterium of a genus overrepresented in tumors compared to normal tumor-adjacent tissue (e.g., a bacterium of a genus listed in Table 3) and/or of a species overrepresented in tumors compared to normal tumor-adjacent tissue (e.g., a bacterium of a species listed in Table 4). In some embodiments, the pharmaceutical composition depletes the tumor of bacteria of two or more genera listed in Table 1 (e.g., at least 3, 4, 5, 6, 7, 8, 9, 10, 11, or 12 of the genera listed in Table 1) and/or two or more species listed in Table 2 (e.g., at least 3, 4, 5, 6, 7, 8, 9, 10, 11, or 12 of the species listed in Table 2). In some embodiments, the pharmaceutical composition comprises an antibiotic. In certain embodiments, administration of the pharmaceutical composition inhibits the growth of the bacteria of bacteria of a genus listed in Table 3 or of a species listed in Table 4. In some embodiments, administration of the pharmaceutical composition kills the bacteria of bacteria of a genus listed in Table 3 or of a species listed in Table 4. In some embodiments, the pharmaceutical composition is administered by intratumoral, subtumoral, and/or peritumoral injection. In some embodiments, administration of the pharmaceutical composition induces tumor cell death. In some embodiments, administration of the pharmaceutical composition induces an anti-tumor immune response.

In certain aspects, provided herein is a method of delivering an agent to a subject with a tumor, the method comprising administering to the subject a pharmaceutical composition comprising bacteria comprising the agent and/or linked to the agent, wherein a bacteria is of a genus overrepresented in tumors compared to normal tumor-adjacent tissue (e.g., bacteria of a genus listed in Table 3) and/or of a species overrepresented in tumors compared to normal tumor-adjacent tissue (e.g., bacteria of a species listed in Table 4). In some embodiments, the agent is covalently linked to the bacteria. In some embodiments, the agent is non-covalently linked to the bacteria (e.g., via a non-covalent lipophilic interaction, an antibody/antigen interaction, a streptavidin/biotin interaction, or a sequence-specific DNA hybridization interaction).

In some aspects, provided herein is a method of delivering an agent to a subject with a tumor, the method comprising administering to the subject a pharmaceutical composition comprising bacteria that express the agent, wherein a bacteria is of a genus overrepresented in tumors compared to normal tumor-adjacent tissue (e.g., bacteria of a genus listed in Table 3) and/or of a species overrepresented in tumors compared to normal tumor-adjacent tissue (e.g., bacteria of a species listed in Table 4). In some embodiments, the bacteria constitutively express the agent. In some embodiments, the bacteria conditionally express the agent (e.g., in response to a quorum sensing switch and/or an environmental change, such as a change in pH, a change in bacterial population density, a change in the environmental oxygen levels and a change in available sugar sources).

In some embodiments of the methods and compositions provided herein, the tumor is selected from the group consisting of acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, neuroblastoma, breast cancer, ovarian cancer, bladder cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma. In some embodiments, the tumor is a colon tumor.

In some embodiments, the methods provided herein further comprise administering to the subject a second cancer therapy. In some embodiments, the second cancer therapy comprises the administration of a chemotherapy agent to the subject. In some embodiments, the second cancer therapy comprises a cancer immunotherapy (e.g., an immune checkpoint inhibitor, a cancer-specific antibody or antigen-binding fragment thereof, a cancer vaccine, an antigen presenting cell (APC) primed with a cancer-specific antigen, a cancer-specific chimeric antigen receptor (CAR), a cancer-specific T cell to the subject, an immune activating protein, an adjuvant). In some embodiments, the second cancer therapy comprises administering an angiogenesis inhibitor to the subject. In some embodiments, the second cancer therapy comprises radiation therapy. In some embodiments, the second cancer therapy comprises administering an antibiotic to the subject. In some embodiments, the second cancer therapy comprises administering to the subject a therapeutic bacteria.

In some embodiments, the subject is a mammal. In some embodiments, the subject is a human. In some embodiments, the subject is a non-human mammal (e.g., a dog, a cat, a cow, a horse, a pig, a donkey, a goat, a camel, a mouse, a rat, a guinea pig, a sheep, a llama, a monkey, a gorilla or a chimpanzee).

### BRIEF DESCRIPTION OF THE DRAWINGS

FIG. 1 shows the log 2(Tumor/Normal Adjacent Tissue) ratio of genera relative abundance.

FIG. 2 shows the log 2(Tumor/Normal Adjacent Tissue) ratio of species relative abundance.

### DETAILED DESCRIPTION

### Definitions

“Adjuvant” or “Adjuvant therapy” broadly refers to an agent that affects an immunological or physiological response in a patient or subject. For example, an adjuvant might increase the presence of an antigen over time or to an area of interest like a tumor, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition to a subject. Examples of routes of administration include oral administration, rectal administration, topical administration, inhalation (nasal) or injection. Administration by injection includes intravenous (IV), intramuscular (IM), intratumoral (IT), subtumoral (ST), peritumoral (PT), and subcutaneous (SC) administration. The pharmaceutical compositions described herein can be administered in any form by any effective route, including but not limited to intratumoral, oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), intravesical, intrapulmonary, intraduodenal, intragastrical, and intrabronchial. In preferred embodiments, the pharmaceutical compositions described herein are administered orally, rectally, intratumorally, topically, intravesically, by injection into or adjacent to a draining lymph node, intravenously, by inhalation or aerosol, or subcutaneously.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as VH) and a heavy chain constant region. Each light chain includes a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

“Cancer” broadly refers to an uncontrolled, abnormal growth of a host's own cells leading to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s),” “neoplasm(s),” and “tumor(s)” are used herein interchangeably. As used herein, “cancer” refers to all types of cancer or neoplasm or malignant tumors including leukemias, carcinomas and sarcomas, whether new or recurring. Specific examples of cancers are: carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state.

The term “epitope” means a protein determinant capable of specific binding to an antibody. Epitopes usually consist of chemically active surface groupings of molecules such as amino acids or sugar side chains. Certain epitopes can be defined by a particular sequence of amino acids to which an antibody is capable of binding.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Mrtin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

“Immunotherapy” is treatment that uses a subject's immune system to treat cancer and includes, for example, checkpoint inhibitors, cancer vaccines, cytokines, cell therapy, CAR-T cells, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold greater after treatment when compared to a pre-treatment state. Properties that may be increased include immune cells, bacterial cells, stromal cells, myeloid derived suppressor cells, fibroblasts, metabolites, and cytokines.

“Innate immune agonists” or “immuno-adjuvants” are small molecules, proteins, or other agents that specifically target innate immune receptors including Toll-Like Receptors, NOD receptors, STING Pathway components. For example, LPS is a TLR-4 agonist that is bacterially derived or synthesized and aluminum can be used as an immune stimulating adjuvant. immuno-adjuvants are a specific class of broader adjuvant or adjuvant therapy.

The term “isolated” or “enriched” encompasses a microbe, bacteria or other entity or substance that has been (1) separated from at least some of the components with which it was associated when initially produced (whether in nature or in an experimental setting), and/or (2) produced, prepared, purified, and/or manufactured by the hand of man. Isolated microbes may be separated from at least about 10%, about 20%, about 30%, about 40%, about 50%, about 60%, about 70%, about 80%, about 90%, or more of the other components with which they were initially associated. In some embodiments, isolated microbes are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a microbe or other material that has been separated from at least some of the components with which it was associated either when initially produced or generated (e.g., whether in nature or in an experimental setting), or during any time after its initial production. A microbe or a microbial population may be considered purified if it is isolated at or after production, such as from a material or environment containing the microbe or microbial population, and a purified microbe or microbial population may contain other materials up to about 10%, about 20%, about 30%, about 40%, about 50%, about 60%, about 70%, about 80%, about 90%, or above about 90% and still be considered “isolated.” In some embodiments, purified microbes or microbial population are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components thereof are generally purified from residual habitat products.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share >97% average nucleotide identity across the entire 16S or some variable region of the 16S are considered the same OTU. See e.g. Claesson M J, Wang Q, O'Sullivan 0, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940. For complete genomes, MLSTs, specific genes, other than 16S, or sets of genes OTUs that share >95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic Glade are provided herein.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

The terms “subject” or “patient” refers to any animal. A subject or a patient described as “in need thereof” refers to one in need of a treatment for a disease. Mammals (i.e., mammalian animals) include humans, laboratory animals (e.g., primates, rats, mice), livestock (e.g., cows, sheep, goats, pigs), and household pets (e.g., dogs, cats, rodents). For example, the subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject or patient may be healthy, or may be suffering from a neoplasm at any developmental stage, wherein any of the stages are either caused by or opportunistically supported of a cancer associated or causative pathogen, or may be at risk of developing a neoplasm, or transmitting to others a cancer associated or cancer causative pathogen. In some embodiments patients have lung cancer, bladder cancer, prostate cancer, ovarian cancer, and/or melanoma. The patients may have tumors that show enhanced macropinocytosis with the underlying genomics of this process including Ras activation. In other embodiments patients suffer from other cancers. In some embodiments, the subject has undergone a cancer therapy.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

As used herein, the term “treating” a disease in a subject or “treating” a subject having or suspected of having a disease refers to subjecting the subject to a pharmaceutical treatment, e.g., the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof.

### Bacteria

In certain aspects, provided herein are methods and compositions related to the treatment of a tumor in a subject by administering to the subject a pharmaceutical composition comprising a bacterium of a genus or species provided in Tables 1 or 2, respectively. In certain aspects, provided herein are methods and compositions related to the treatment of a tumor in a subject by administering to the subject a pharmaceutical composition that depletes the tumor of a bacterium of a genus or species provided in Tables 3 or 4, respectively. In other aspects, the methods and compositions provided herein relate to the use of bacteria of a genus or species provided in Tables 3 and 4, respectively, for the delivery of a therapeutic agent to a tumor.

TABLE 1
Genera of exemplary bacteria underrepresented in tumor samples

| | Log2(Tumor/Normal adjacent tissue) ratio |
|---|---|
| Genus | of relative abundance |
| Intestinimonas | −2.284707805 |
| Ruminiclostridium | −1.685705393 |
| Cedecea | −1.531407936 |
| Parasutterella | −1.492805923 |
| Eggerthella | −1.371695538 |
| Intestinibacter | −1.362028536 |
| Klebsiella | −1.351470093 |
| Faecalibacterium | −1.332105411 |
| Citrobacter | −1.206400321 |
| Candidatus Accumulibacter | −1.162189847 |
| Bifidobacterium | −1.073290635 |
| Erwinia | −1.012819042 |
| Caloramator | −0.943875539 |
| Collinsella | −0.905734926 |
| Shigella | −0.900712516 |
| Subdoligranulum | −0.881119051 |
| Senegalimassilia | −0.862972432 |
| Odoribacter | −0.835876244 |
| Geothrix | −0.808654234 |
| Desulfovibrio | −0.776104693 |
| Phascolarctobacterium | −0.763727121 |
| Oscillibacter | −0.750544985 |
| Tannerella | −0.748811318 |
| Fusicatenibacter | −0.72236771 |
| Lachnobacterium | −0.718531846 |
| Alistipes | −0.7096927 |
| Faecahcoccus | −0.652060994 |
| Mageeibacillus | −0.645658001 |
| Anaerostipes | −0.623864107 |
| Tyzzerella | −0.616731504 |
| Megasphaera | −0.612288781 |
| Ehrlichia | −0.60657176 |
| Parabacteroides | −0.576197591 |
| Niabella | −0.572016566 |
| Butyrivibrio | −0.53701227 |
| Selenomonas | −0.533728241 |
| Bacteroides | −0.52361513 |
| Lactonifactor | −0.508486566 |
| Brachybacterium | −0.507840798 |
| Johnsonella | −0.505490535 |
| Lachnoclostridium | −0.50276958 |

TABLE 2
Species of exemplary bacteria underrepresented in tumor samples

| | Log2(Tumor/Normal adjacent |
|---|---|
| Species | tissue) ratio of relative abundance |
| Prevotella copri | −2.392303328 |
| Acinetobacter radioresistens | −2.169142216 |
| Intestinimonas butyriciproducens | −1.676833198 |
| Faecalibacterium prausnitzii | −1.413715736 |
| Klebsiella oxytoca | −1.288171616 |
| Cronobacter muytjensii | −1.260473988 |
| Parasutterella excrementihominis | −1.257368963 |
| Bacillus firmus | −1.244576024 |
| Clostridium innocuum | −1.147806109 |
| Intestinibacter bartlettii | −1.145903712 |
| Caloramator fervidus | −1.093255924 |
| Tyzzerella nexilis | −1.039748448 |
| Clostridium leptum | −1.02447752 |
| Streptococcus downei | −1.02103523 |

TABLE 3
Genera of exemplary bacteria overrepresented in tumor samples

| | Log2(Tumor/Normal adjacent tissue) ratio |
|---|---|
| Genus | of relative abundance |
| Necropsobacter | 5.437330569 |
| Leptotrichia | 3.075575322 |
| Carnobacterium | 2.941305755 |
| Fusobacterium | 2.708018791 |
| Alkalibacter | 2.323592289 |
| Veillonella | 2.04874144 |
| Pediococcus | 1.48937536 |
| Campylobacter | 1.275359119 |
| Aggregatibacter | 1.268284719 |
| Bosea | 1.071353661 |
| Pectobacterium | 1.044111153 |
| Lactococcus | 1.036590381 |
| Enterococcus | 0.923888944 |
| Cetobacterium | 0.845205176 |
| Psychrobacter | 0.837286245 |
| Aeromonas | 0.789764873 |
| Streptococcus | 0.776434283 |
| Aerococcus | 0.755324582 |
| Slackia | 0.693386099 |
| Pseudomonas | 0.631776755 |
| Arsenophonus | 0.601418096 |
| Plesiomonas | 0.593696557 |
| Dickeya | 0.564005542 |
| Salinispirillum | 0.535501338 |

TABLE 4
Species of exemplary bacteria overrepresented in tumor samples

| | Log2(Tumor/Normal adjacent |
|---|---|
| Species | tissue) ratio of relative abundance |
| Leptotrichia wadei | 8.218422828 |
| Oxalophagus oxalicus | 6.069851281 |
| Streptococcus peroris | 6.069851281 |
| Bacillus vallismortis | 5.927945465 |
| Necropsobacter rosorum | 5.927945465 |
| Streptococcus infantarius | 5.927945465 |
| Veillonella montpellierensis | 5.462487843 |
| Halorhodospira neutriphila | 5.279729161 |
| Aerococcus viridans | 4.948485215 |
| Staphylococcus epidermidis | 4.629460558 |
| Lactobacillus sakei | 4.543120313 |
| Fusobacterium periodonticum | 4.221579456 |
| Fusobacterium gonidiaformans | 3.486097245 |
| Carnobacterium maltaromaticum | 3.443169071 |
| Bacillus chungangensis | 3.116021113 |
| Aminomonas paucivorans | 3.053074558 |
| Leptotrichia goodfellowii | 2.982779358 |
| Alkalibacter saccharofermentans | 2.880300273 |
| Fusobacterium naviforme | 2.574890834 |
| Streptococcus sanguinis | 2.425908265 |
| Gemella palaticanis | 2.272990887 |
| Fusobacterium hwasookii | 2.213656605 |
| Fusobacterium mortiferum | 2.108481552 |
| Streptococcus dysgalactiae | 2.104707528 |
| Veillonella caviae | 2.061545801 |
| Streptococcus cristatus | 2.035720534 |
| Photorhabdus luminescens | 1.980679915 |
| Streptococcus gallolyticus | 1.955027602 |
| Veillonella parvula | 1.915868805 |
| Pediococcus pentosaceus | 1.906887396 |
| Leptotrichia trevisanii | 1.865511845 |
| Streptococcus dentasini | 1.859251192 |
| Fusobacterium sp. oral taxon 203 | 1.851852598 |
| Streptococcus sp. oral taxon 071 | 1.840885698 |
| Streptococcus mitis | 1.788066954 |
| Filifactor alocis | 1.781219432 |
| Pectobacterium carotovorum | 1.73133652 |
| Enterococcus hirae | 1.69081351 |
| Erwinia toletana | 1.69081351 |
| Proteus vulgaris | 1.69081351 |
| Streptococcus intermedius | 1.659003977 |
| Oribacterium asaccharolyticum | 1.654543768 |
| Lactobacillus casei | 1.636871966 |
| Streptococcus gordonii | 1.564511987 |
| Streptococcus iniae | 1.558441541 |
| Desulfotomaculum putei | 1.553070404 |
| Enterococcus gallinarum | 1.549746283 |
| Clostridium tertium | 1.542940612 |
| Streptococcus parauberis | 1.528389312 |
| Streptococcus macedonicus | 1.506139755 |
| Fusobacterium equinum | 1.401902283 |
| Wollea vaginicola | 1.401902283 |
| Megamonas hypermegale | 1.397036795 |
| Weissella confusa | 1.385731556 |
| Eremococcus coleocola | 1.380311769 |
| Haemophilus haemolyticus | 1.380311769 |
| Cronobacter dublinensis | 1.360001104 |
| Streptococcus equinus | 1.351571278 |
| Enterobacter hormaechei | 1.314652437 |
| Streptococcus thermophilus | 1.291726589 |
| Trueperella pyogenes | 1.290554544 |
| Bacillus tequilensis | 1.290437195 |
| Gilliamella apicola | 1.276974347 |
| Bacillus flexus | 1.250312964 |
| Psychrobacter urativorans | 1.122210474 |
| Streptococcus pyogenes | 1.119749665 |
| Streptococcus entericus | 1.115759512 |
| Lactobacillus rhamnosus | 1.113958429 |
| Allobaculum stercoricanis | 1.112547931 |
| Facklamia tabacinasalis | 1.112547931 |
| Psychrobacter pulmonis | 1.102570704 |
| Enterococcus faecium | 1.098232927 |
| Streptococcus parasanguinis | 1.097981365 |
| Enterococcus lactis | 1.081327408 |
| Gallibacterium anatis | 1.064347567 |
| Lactobacillus algidus | 1.063359145 |
| Tetragenococcus muriaticus | 1.060224421 |
| Aeromonas sharmana | 1.05552027 |
| Enterobacter aerogenes | 1.05552027 |
| Lactobacillus mellifer | 1.05552027 |
| Bosea thiooxidans | 1.048934123 |
| Anaerolinea thermolimosa | 1.041086042 |
| Streptococcus didelphis | 1.014935436 |

In some embodiments, the bacteria used in the methods provided herein are engineered (modified) microbes. For example, engineered microbes include microbes harboring i) one or more genetic changes, such change being an insertion, deletion, translocation, or substitution, or any combination thereof, of one or more nucleotides contained on the bacterial chromosome or on an endogenous plasmid, wherein the genetic change may result in the alteration, disruption, removal, or addition of one or more protein coding genes, non-protein-coding genes, gene regulatory regions, or any combination thereof, and wherein such change may be a fusion of two or more separate genomic regions or may be synthetically derived; ii) one or more foreign plasmids containing a mutant copy of an endogenous gene, such mutation being an insertion, deletion, or substitution, or any combination thereof, of one or more nucleotides; iii) one or more foreign plasmids containing a mutant or non-mutant exogenous gene or a fusion of two or more endogenous, exogenous, or mixed genes; iv) one or more chemotherapeutic agents, or v) chromosome and/or plasmid-encoded genes for the production of one or more anti-cancer chemicals, including but not limited to cancer-associated antigens, radionuclides, and chemotherapeutic agents. The engineered microbe(s) may be produced using techniques including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, or any combination thereof. Suitable microbes for engineering are known in the art.

In certain embodiments, the bacterial composition comprises killed, live, and/or attenuated bacteria.

### Bacterial Formulations

In certain embodiments, the methods provided herein include the step of administering a pharmaceutical composition comprising a bacterium and/or a combination of bacteria to a subject. In certain embodiments, the bacterium is administered to the subject in a pharmaceutical composition (i.e., a bacterial composition). In some embodiments, the bacterial formulation comprises a bacterium and/or a combination of bacteria described herein and a pharmaceutically acceptable carrier.

In certain embodiments, at least 50%, 55%, 60%, 65%, 70%, 75%, 80%, 85%, 90%, 95%, 96%, 97%, 98%, 99% of the bacteria in the bacterial composition are of a genus listed in Table 1 or 3 or of a species listed in Table 2 or 4. In certain embodiments, substantially all of the bacteria in the bacterial composition are of a genus listed in Table 1 or 3 or of a species listed in Table 2 or 4.

In certain embodiments, the bacterial composition comprises at least 1×10³ colony forming units (CFUs), 1×10⁴ colony forming units (CFUs), 1×10⁵ colony forming units (CFUs), 5×10⁵ colony forming units (CFUs), 1×10⁶ colony forming units (CFUs), 2×10⁶ colony forming units (CFUs), 3×10⁶ colony forming units (CFUs), 4×10⁶ colony forming units (CFUs), 5×10⁶ colony forming units (CFUs), 6×10⁶ colony forming units (CFUs), 7×10⁶ colony forming units (CFUs), 8×10⁶ colony forming units (CFUs), 9×10⁶ colony forming units (CFUs), 1×10⁷ colony forming units (CFUs), 2×10⁷ colony forming units (CFUs), 3×10⁷ colony forming units (CFUs), 4×10⁷ colony forming units (CFUs), 5×10⁷ colony forming units (CFUs), 6×10⁷ colony forming units (CFUs), 7×10⁷ colony forming units (CFUs), 8×10⁷ colony forming units (CFUs), 9×10⁷ colony forming units (CFUs), 1×10⁸ colony forming units (CFUs), 2×10⁸ colony forming units (CFUs), 3×10⁸ colony forming units (CFUs), 4×10⁸ colony forming units (CFUs), 5×10⁸ colony forming units (CFUs), 6×10⁸ colony forming units (CFUs), 7×10⁸ colony forming units (CFUs), 8×10⁸ colony forming units (CFUs), 9×10⁸ colony forming units (CFUs), 1×10⁹ colony forming units (CFUs), 5×10⁹ colony forming units (CFUs), 1×10¹⁰ colony forming units (CFUs) of bacteria in the pharmaceutical formulation are bacteria of a genus listed in Table 1 or 3 or of a species listed in Table 2 or 4.

Methods for producing bacteria may include three main processing steps. The steps are: organism banking, organism production, and preservation.

For banking, the strains included in the bacteria may be (1) isolated directly from a specimen or taken from a banked stock, (2) optionally cultured on a nutrient agar or broth that supports growth to generate viable biomass, and (3) the biomass optionally preserved in multiple aliquots in long-term storage.

In embodiments using a culturing step, the agar or broth may contain nutrients that provide essential elements and specific factors that enable growth. An example would be a medium composed of 20 g/L glucose, 10 g/L yeast extract, 10 g/L soy peptone, 2 g/L citric acid, 1.5 g/L sodium phosphate monobasic, 100 mg/L ferric ammonium citrate, 80 mg/L magnesium sulfate, 10 mg/L hemin chloride, 2 mg/L calcium chloride, 1 mg/L menadione. Another examples would be a medium composed of 10 g/L beef extract, 10 g/L peptone, 5 g/L sodium chloride, 5 g/L dextrose, 3 g/L yeast extract, 3 g/L sodium acetate, 1 g/L soluble starch, and 0.5 g/L L-cysteine HCl, at pH 6.8. A variety of microbiological media and variations are well known in the art (e.g., R. M. Atlas, Handbook of Microbiological Media (2010) CRC Press). Culture media can be added to the culture at the start, may be added during the culture, or may be intermittently/continuously flowed through the culture. The strains in the bacterial composition may be cultivated alone, as a subset of the microbial composition, or as an entire collection comprising the microbial composition. As an example, a first strain may be cultivated together with a second strain in a mixed continuous culture, at a dilution rate lower than the maximum growth rate of either cell to prevent the culture from washing out of the cultivation.

The inoculated culture is incubated under favorable conditions for a time sufficient to build biomass. For microbial compositions for human use this is often at 37° C. temperature, pH, and other parameter with values similar to the normal human niche. The environment may be actively controlled, passively controlled (e.g., via buffers), or allowed to drift. For example, for anaerobic bacterial compositions, an anoxic/reducing environment may be employed. This can be accomplished by addition of reducing agents such as cysteine to the broth, and/or stripping it of oxygen. As an example, a culture of a bacterial composition may be grown at 37° C., pH 7, in the medium above, pre-reduced with 1 g/L cysteine-HCl.

When the culture has generated sufficient biomass, it may be preserved for banking. The organisms may be placed into a chemical milieu that protects from freezing (adding ‘cryoprotectants’), drying (‘lyoprotectants’), and/or osmotic shock (‘osmoprotectants’), dispensing into multiple (optionally identical) containers to create a uniform bank, and then treating the culture for preservation. Containers are generally impermeable and have closures that assure isolation from the environment. Cryopreservation treatment is accomplished by freezing a liquid at ultra-low temperatures (e.g., at or below −80° C.). Dried preservation removes water from the culture by evaporation (in the case of spray drying or ‘cool drying’) or by sublimation (e.g., for freeze drying, spray freeze drying). Removal of water improves long-term microbial composition storage stability at temperatures elevated above cryogenic. If the microbial composition comprises, for example, spore forming species and results in the production of spores, the final composition may be purified by additional means such as density gradient centrifugation preserved using the techniques described above. Microbial composition banking may be done by culturing and preserving the strains individually, or by mixing the strains together to create a combined bank. As an example of cryopreservation, a microbial composition culture may be harvested by centrifugation to pellet the cells from the culture medium, the supernatant decanted and replaced with fresh culture broth containing 15% glycerol. The culture can then be aliquoted into 1 mL cryotubes, sealed, and placed at −80° C. for long-term viability retention. This procedure achieves acceptable viability upon recovery from frozen storage.

Microbial production may be conducted using similar culture steps to banking, including medium composition and culture conditions. It may be conducted at larger scales of operation, especially for clinical development or commercial production. At larger scales, there may be several subcultivations of the microbial composition prior to the final cultivation. At the end of cultivation, the culture is harvested to enable further formulation into a dosage form for administration. This can involve concentration, removal of undesirable medium components, and/or introduction into a chemical milieu that preserves the microbial composition and renders it acceptable for administration via the chosen route. For example, a microbial composition may be cultivated to a concentration of 10¹⁰ CFU/mL, then concentrated 20-fold by tangential flow microfiltration; the spent medium may be exchanged by diafiltering with a preservative medium consisting of 2% gelatin, 100 mM trehalose, and 10 mM sodium phosphate buffer. The suspension can then be freeze-dried to a powder and titrated.

After drying, the powder may be blended to an appropriate potency, and mixed with other cultures and/or a filler such as microcrystalline cellulose for consistency and ease of handling, and the bacterial composition formulated as provided herein.

In certain aspects, provided are bacterial compositions for administration subjects. In some embodiments, the bacterial compositions are combined with additional active and/or inactive materials in order to produce a final product, which may be in single dosage unit or in a multi-dose format.

In some embodiments the composition comprises at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

In some embodiments the composition comprises at least one lipid. As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments the composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments the excipient is a buffering agent. Non-limiting examples of suitable buffering agents include sodium citrate, magnesium carbonate, magnesium bicarbonate, calcium carbonate, and calcium bicarbonate.

In some embodiments the excipient comprises a preservative. Non-limiting examples of suitable preservatives include antioxidants, such as alpha-tocopherol and ascorbate, and antimicrobials, such as parabens, chlorobutanol, and phenol.

In some embodiments the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments the composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In certain embodiments, the bacteria disclosed herein are administered in conjunction with a prebiotic to the subject. Prebiotics are carbohydrates which are generally indigestible by a host animal and are selectively fermented or metabolized by bacteria. Prebiotics may be short-chain carbohydrates (e.g., oligosaccharides) and/or simple sugars (e.g., mono- and di-saccharides) and/or mucins (heavily glycosylated proteins) that alter the composition or metabolism of a microbiome in the host. The short chain carbohydrates are also referred to as oligosaccharides, and usually contain from 2 or 3 and up to 8, 9, 10, 15 or more sugar moieties. When prebiotics are introduced to a host, the prebiotics affect the bacteria within the host and do not directly affect the host. In certain aspects, a prebiotic composition can selectively stimulate the growth and/or activity of one of a limited number of bacteria in a host. Prebiotics include oligosaccharides such as fructooligosaccharides (FOS) (including inulin), galactooligosaccharides (GOS), trans-galactooligosaccharides, xylooligosaccharides (XOS), chitooligosaccharides (COS), soy oligosaccharides (e.g., stachyose and raffinose) gentiooligosaccharides, isomaltooligosaccharides, mannooligosaccharides, maltooligosaccharides and mannanoligosaccharides. Oligosaccharides are not necessarily single components, and can be mixtures containing oligosaccharides with different degrees of oligomerization, sometimes including the parent disaccharide and the monomeric sugars. Various types of oligosaccharides are found as natural components in many common foods, including fruits, vegetables, milk, and honey. Specific examples of oligosaccharides are lactulose, lactosucrose, palatinose, glycosyl sucrose, guar gum, gum Arabic, tagalose, amylose, amylopectin, pectin, xylan, and cyclodextrins. Prebiotics may also be purified or chemically or enzymatically synthesized.

### Administration

In certain aspects, provided herein is a method of delivering a bacterium described herein to a subject. In some embodiments, administration of the pharmaceutical composition induces tumor cell death. In some embodiments, administration of the pharmaceutical composition induces an anti-tumor immune response.

In some embodiments of the methods provided herein, the bacteria are administered in conjunction with the administration of an antibiotic. In some embodiments, the bacteria is co-formulated in a pharmaceutical composition with the antibiotic. In some embodiments, the bacteria is co-administered with the antibiotic. In some embodiments, the antibiotic is administered to the subject before administration of the bacteria (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the antibiotic is administered to the subject after administration of the bacteria (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments the same mode of delivery are used to deliver both the bacteria and the antibiotic. In some embodiments different modes of delivery are used to administer the bacteria and the antibiotic. For example, in some embodiments the antibiotic administered orally while the bacteria is administered via injection (e.g., an intravenous, intramuscular, subtumoral, peritumoral, and/or intratumoral injection).

In some embodiments of the methods provided herein, the bacteria are administered in conjunction with the administration of a cancer therapeutic. In some embodiments, the bacteria is co-formulated in a pharmaceutical composition with the cancer therapeutic. In some embodiments, the bacteria is co-administered with the cancer therapeutic. In some embodiments, the cancer therapeutic is administered to the subject before administration of the bacteria (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the cancer therapeutic is administered to the subject after administration of the bacteria (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments the same mode of delivery are used to deliver both the bacteria and the cancer therapeutic. In some embodiments different modes of delivery are used to administer the bacteria and the cancer therapeutic. For example, in some embodiments the bacteria administered orally while the cancer therapeutic is administered via injection (e.g., an intravenous, intramuscular, subtumoral, peritumoral, and/or intratumoral injection).

In certain embodiments, the pharmaceutical compositions, dosage forms, and kits described herein can be administered in conjunction with any other conventional anti-cancer treatment, such as, for example, radiation therapy and surgical resection of the tumor. These treatments may be applied as necessary and/or as indicated and may occur before, concurrent with or after administration of the pharmaceutical compositions, dosage forms, and kits described herein.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, for example, tumor size, and other compounds such as drugs being administered concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate in a tumor or metastasis. The methods of treatment described herein may be suitable for the treatment of a primary tumor, a secondary tumor or metastasis, as well as for recurring tumors or cancers. The dose of the pharmaceutical compositions described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent cancer, delay its onset, or slow or stop its progression. One skilled in the art will recognize that dosage will depend upon a variety of factors including the strength of the particular compound employed, as well as the age, species, condition, and body weight of the subject. The size of the dose will also be determined by the route, timing, and frequency of administration as well as the existence, nature, and extent of any adverse side-effects that might accompany the administration of a particular compound and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the active agents used in accordance with the invention vary depending on the active agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and most preferably causing complete regression of the cancer.

Separate administrations can include any number of two or more administrations (e.g., doses), including two, three, four, five or six administrations. One skilled in the art can readily determine the number of administrations to perform or the desirability of performing one or more additional administrations according to methods known in the art for monitoring therapeutic methods and other monitoring methods provided herein. In some embodiments, the doses may be separated by at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days or 1, 2, 3, or 4 weeks. Accordingly, the methods provided herein include methods of providing to the subject one or more administrations of a bacterium, where the number of administrations can be determined by monitoring the subject, and, based on the results of the monitoring, determining whether or not to provide one or more additional administrations. Deciding on whether or not to provide one or more additional administrations can be based on a variety of monitoring results, including, but not limited to, indication of tumor growth or inhibition of tumor growth, appearance of new metastases or inhibition of metastasis, the subject's anti-bacterium antibody titer, the subject's anti-tumor antibody titer, the overall health of the subject and/or the weight of the subject.

The time period between administrations can be any of a variety of time periods. The time period between administrations can be a function of any of a variety of factors, including monitoring steps, as described in relation to the number of administrations, the time period for a subject to mount an immune response and/or the time period for a subject to clear the bacteria from normal tissue. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the bacteria from normal tissue; for example, the time period can be more than the time period for a subject to clear the bacteria from normal tissue, such as more than about a day, more than about two days, more than about three days, more than about five days, or more than about a week.

In some embodiments, the delivery of a cancer therapeutics in combination with the bacteria described herein reduces the adverse effects and/or improves the efficacy of the cancer therapeutic.

The effective dose of a cancer therapeutic described herein is the amount of the therapeutic agent that is effective to achieve the desired therapeutic response for a particular patient, composition, and mode of administration, with the least toxicity to the patient. The effective dosage level can be identified using the methods described herein and will depend upon a variety of pharmacokinetic factors including the activity of the particular compositions administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. In general, an effective dose of a cancer therapy will be the amount of the therapeutic agent which is the lowest dose effective to produce a therapeutic effect. Such an effective dose will generally depend upon the factors described above.

The toxicity of a cancer therapy is the level of adverse effects experienced by the subject during and following treatment. Adverse events associated with cancer therapy toxicity include, but are not limited to, abdominal pain, acid indigestion, acid reflux, allergic reactions, alopecia, anaphylaxis, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypernatremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heart beat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

### Depletion of Tumor-Associated Bacteria

In certain aspects, the methods provided herein include methods comprising depleting a tumor of bacteria. In other aspects, the methods provide herein change a tumor microbiome by depleting the tumor of bacteria. In certain other aspects, the methods provided herein include administering a pharmaceutical composition to the subject that selectively depletes the tumor of bacteria. In some embodiments, the pharmaceutical composition comprises an antibiotic. In some embodiemts, the methods provided herein include administering a vaccine to the subject that selectively depletes the tumor of bacteria.

In some embodiments, the depleted bacterium is of a genus listed in Table 3. In some embodiments, the depleted bacterium is of a species listed in Table 4.

In some embodiments, a vaccine is administered to the subject that induces an immune response against the bacteria to be depleted. In some embodiments, the administration of the vaccine inhibits the growth of the bacteria. In certain embodiments, the administration of the vaccine kills the bacteria. In some embodiments, the vaccine comprises live, attenuated, or killed bacteria (e.g., bacteria of a genus listed in Table 3 or of a species listed in Table 4). In some embodiments, the vaccine comprises a component of a bacteria (e.g., a bacterial peptide and/or protein, bacterial DNA or RNA). In some embodiments, the vaccine is a protein vaccine, a nucleic acid vaccine or a combination thereof. For example, in some embodiments, the vaccine comprises a polypeptide comprising an epitope of a bacterial antigen. In some embodiments, the cancer vaccine comprises a nucleic acid (e.g., DNA or RNA, such as mRNA) that encodes an epitope of a bacterial antigen. In some embdoiments, the vaccine elicits an immune response to the bacteria.

In some embodiments, the vaccine is administered with an adjuvant. Examples of adjuvants include, but are not limited to, an immune modulatory protein, Adjuvant 65, α-GalCer, aluminum phosphate, aluminum hydroxide, calcium phosphate, 3-Glucan Peptide, CpG DNA, GPI-0100, lipid A, lipopolysaccharide, Lipovant, Montanide, N-acetyl-muramyl-L-alanyl-D-isoglutamine, Pam3CSK4, quil A and trehalose dimycolate.

In some embodiments, the administration of the pharmaceutical composition inhibits the growth of the bacteria of the bacteria. In certain embodiments, the administration of the pharmaceutical composition kills the bacteria.

In certain embodiments of the methods disclose herein, the pharmaceutical composition comprises an antibiotic. “Antibiotics” broadly refers to compounds capable of inhibiting or preventing a bacterial infection. Antibiotics can be classified in a number of ways, including their use for specific infections, their mechanism of action, their bioavailability, or their spectrum of target microbe (e.g., Gram-negative vs. Gram-positive bacteria, aerobic vs. anaerobic bacteria, etc.) and these may be used to kill specific bacteria in specific areas of the host (“niches”) (Leekha, et al 2011. General Principles of Antimicrobial Therapy. Mayo Clin Proc. 86(2): 156-167). In certain embodiments, antibiotics can be used to selectively target bacteria of a specific niche. In some embodiments, antibiotics known to treat a particular infection that includes a cancer niche may be used to target cancer-associated microbes, including cancer-associated bacteria in that niche. In other embodiments, antibiotics are administered after the bacterial treatment. In some embodiments, antibiotics are administered after the bacterial treatment to remove the engraftment.

In some aspects, antibiotics can be selected based on their bactericidal or bacteriostatic properties. Bactericidal antibiotics include mechanisms of action that disrupt the cell wall (e.g., β-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin. Aminoglycosides are effective, e.g., against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin. Geldanamycin and Herbimycin are believed to inhibit or alter the function of Heat Shock Protein 90.

Carbacephems include, but are not limited to, Loracarbef. Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, e.g., against anaerobic bacteria, as well as Staphylococcus, and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin. Penicillins are effective, e.g., against Gram-positive bacteria, facultative anaerobes, e.g., Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to, Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin Pl, clarithromycin, erythromycins, furazolidone, fusidic acid, Na fusidate, gramicidin, imipenem, indolicidin, josamycin, magainan II, metronidazole, nitroimidazoles, mikamycin, mutacin B-Ny266, mutacin B-JH1 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

### Targeting Agents to Tumors Using Tumor-Associated Bacteria

In some embodiments, the bacteria provided herein (e.g., bacteria of a genus listed in Table 3 and/or of a species listed in Table 4) have an infection profile wherein they localize to and/or infect certain cells, tissues and/or organs (e.g., tumors, such as colon tumors). In some embodiments, the bacteria are modified (e.g., modified using a method provided herein) to have a particular infection profile. In some embodiments, the bacteria used in the methods provided herein are selected, at least in part, because of their infection profile. In some embodiments, the bacteria have an infection profile in which they localize to tumors and/or cancer cells. In some embodiments, the bacteria have an infection profile where they localize to a particular organ and/or tissue (e.g., colon). In some embodiments, the bacteria have an infection profile wherein they infect and/or bind to particular cell types. In some embodiments, the bacteria have an infection profile wherein they infect and/or bind to cancer and/or tumor cells. In some embodiments, the bacteria have an infection profile wherein they infect and/or bind to antigen presenting cells (e.g., dendritic cells, macrophages, B cells). In some embodiments, the bacteria have and infection profile wherein they infect and/or bind to tumor-associated macrophages and/or myeloid-derived suppressor cells.

In certain embodiments, the bacteria provided herein are selected and/or modified to comprise genes that are conditionally expressed when the bacteria are exposed to certain environmental conditions (e.g., low pH, low oxygen, high lactate concentration). In some embodiments, the conditionally expressed gene is operably linked to a low-pH induced promoter, such as STM1787. In some embodiments, the conditionally expressed gene is operably linked to a hypoxia-induced promoter, such as pepT, pflE, ansB, vhb or FF+20*. In some embodiments, the gene encodes a cancer therapeutic described herein. In some embodiments, the gene encodes a prodrug enzyme described herein. In some embodiments, the gene encodes a protein that causes the lysis of the bacteria. In some embodiments, such bacteria comprise a cancer therapeutic and/or a prodrug enzyme described herein that is released upon lysis of the bacteria.

In some embodiments, the bacteria are quorum-sensing bacteria. Quorum-sensing allows bacteria to measure the density of their local population and adjust gene expression depending on the cell density. Thus, in some embodiments, the quorum-sensing bacteria comprise a gene that is conditionally expressed when by the bacteria described herein when the bacteria are present at a certain density (e.g., at a tumor). In some embodiments, the conditionally expressed gene is under the control of the p(luxI) promoter, as described in, for example, Swofford C. A., et al., Proc. Natl. Acad. Sci. USA, 2015, 112(11):3457-62, which is hereby incorporated by reference in its entirety. In some embodiments, the gene is expressed when the bacteria reach a cell density of 1 CFU/ml, 10 CFU/ml, 100 CFU/ml, 1×10⁴ CFU/ml, 1×10⁵ CFU/ml, 1×10⁶ CFU/ml, 1×10⁷ CFU/ml, 1×10⁸ CFU/ml, 1×10⁹ CFU/ml, or 1×10¹⁰ CFU/ml.

In some embodiments, the bacteria described herein are modified such that they release cancer therapeutic agents after a time delay. In some embodiments, the time delay is the result of an inhibition of a bacterial efflux pump in the bacteria. In some embodiments, the bacteria comprise a small molecule cancer therapeutic that is capable of being extruded by a bacterial efflux pump. In some embodiments, the function of the bacterial efflux pumps of the bacteria are inhibited (e.g., using a covalent inhibitor) such that the bacteria are not able to extrude the cancer therapeutic until new efflux pumps are generated by the bacteria.

In some embodiments, the bacteria described herein are modified such that the release of a cancer therapeutic by the bacteria is facilitated by the presence of a second modified bacteria. For example, in some embodiments, a cancer therapeutic (e.g., the anti-cancer drug doxorubicin) is attached to the bacteria by a double-stranded nucleic acid that comprises a cleavage site (e.g., a restriction site and/or a zinc finger nuclease target site. The modified bacteria can then be administered to a subject in conjunction with a second bacteria that localizes to a tumor and that expresses and/or is bound by a the restriction enzyme or zinc finger nuclease that is able to cleave the nucleic acid linker. When the bacteria described herein and the second bacteria co-localize to the tumor, the nucleic acid linker is cleaved and the cancer therapeutic is released.

In some embodiments, the bacteria provided herein are bound to a cancer therapeutic by a cross-linker. As used herein, the term “cross-linker” broadly refers to compositions that can be used to join various molecules, including proteins, together. Examples of cross-linkers include, but are not limited to, 1,5-difluoro-2,4-dinitrobenzene, 3,3′-dithiobis(succinimidyl propionate), bis(2-succinimidooxycarbonyloxy)ethyl)sulfone, bis(sulfosuccinimidyl)suberate, dimethyl 3,3′-dithiobispropionimidate, dimethyl adipimidate, dimethyl pimelimidate, dimethyl suberimidate, disuccinimidyl glutarate, disuccinimidyl suberate, disuccinimidyl tartrate, dithiobis(succinimidyl propionate), ethylene glycosl bis(succinimidyl succinate), ethylene glycosl bis(sulfosuccinimidyl succinate), PEGylated bis(sulfosuccinimidyl)suberate (with PEG5), PEGylated bis(sulfosuccinimidyl)suberate (with PEG9), and tris-(succinimidyl)aminotriacetate, as well as those listed in Table 5, below.

TABLE 5
Exemplary Cross-Linkers

| Crosslinker | Crosslinking Target |
|---|---|
| BS(PEG)5 | Amine-to-Amine |
| BS(PEG)9 | Amine-to-Amine |
| BS3 | Amine-to-Amine |
| BSOCOES | Amine-to-Amine |
| DFDNB | Amine-to-Amine |
| DMA | Amine-to-Amine |
| DMP | Amine-to-Amine |
| DMS | Amine-to-Amine |
| DSG | Amine-to-Amine |
| DSP | Amine-to-Amine |
| DSS | Amine-to-Amine |
| DST | Amine-to-Amine |
| DTBP | Amine-to-Amine |
| DTSSP | Amine-to-Amine |
| EGS | Amine-to-Amine |
| Sulfo-EGS | Amine-to-Amine |
| TSAT | Amine-to-Amine |
| AMAS | Amine-to-Sulfhydryl |
| BMPS | Amine-to-Sulfhydryl |
| EMCS | Amine-to-Sulfhydryl |
| GMBS | Amine-to-Sulfhydryl |
| LC-SMCC | Amine-to-Sulfhydryl |
| LC-SPDP | Amine-to-Sulfhydryl |
| MBS | Amine-to-Sulfhydryl |
| PEG12-SPDP | Amine-to-Sulfhydryl |
| PEG4-SPDP | Amine-to-Sulfhydryl |
| SBAP | Amine-to-Sulfhydryl |
| SIA | Amine-to-Sulfhydryl |
| SIAB | Amine-to-Sulfhydryl |
| SM(PEG)12 | Amine-to-Sulfhydryl |
| SM(PEG)2 | Amine-to-Sulfhydryl |
| SM(PEG)24 | Amine-to-Sulfhydryl |
| SM(PEG)4 | Amine-to-Sulfhydryl |
| SM(PEG)6 | Amine-to-Sulfhydryl |
| SM(PEG)8 | Amine-to-Sulfhydryl |
| SMCC | Amine-to-Sulfhydryl |
| SMPH | Amine-to-Sulfhydryl |
| SMPT | Amine-to-Sulfhydryl |
| SPDP | Amine-to-Sulfhydryl |
| Sulfo-EMCS | Amine-to-Sulfhydryl |
| Sulfo-GMBS | Amine-to-Sulfhydryl |
| Sulfo-KMUS | Amine-to-Sulfhydryl |
| Sulfo-LC-SPDP | Amine-to-Sulfhydryl |
| Sulfo-MBS | Amine-to-Sulfhydryl |
| Sulfo-SIAB | Amine-to-Sulfhydryl |
| Sulfo-SMCC | Amine-to-Sulfhydryl |
| DCC | Carboxyl-to-Amine |
| EDC | Carboxyl-to-Amine |
| NHS | Carboxyl-to-Amine |
| Sulfo-NHS | Carboxyl-to-Amine |
| BMPH | Sulfhydryl-to-Carbohydrate |
| EMCH | Sulfhydryl-to-Carbohydrate |
| KMUH | Sulfhydryl-to-Carbohydrate |
| MPBH | Sulfhydryl-to-Carbohydrate |
| PDPH | Sulfhydryl-to-Carbohydrate |
| BM(PEG)2 | Sulfhydryl-to-Sulfhydryl |
| BM(PEG)3 | Sulfhydryl-to-Sulfhydryl |
| BMB | Sulfhydryl-to-Sulfhydryl |
| BMH | Sulfhydryl-to-Sulfhydryl |
| BMOE | Sulfhydryl-to-Sulfhydryl |
| DTME | Sulfhydryl-to-Sulfhydryl |
| TMEA | Sulfhydryl-to-Sulfhydryl |
| ANB-NOS | Photoreactive |
| LC-SDA | Photoreactive |
| SDA | Photoreactive |
| SDAD | Photoreactive |
| Sulfo-LC-SDA | Photoreactive |
| Sulfo-SANPAH | Photoreactive |
| Sulfo-SDA | Photoreactive |
| Sulfo-SDAD | Photoreactive |

In some embodiments, the bacteria described herein is linked to a cancer therapeutic through a nucleic acid linker. For example, in some embodiments, the bacteria described herein display a first single-stranded nucleic acid oligonucleotide (e.g., an oligonucleotide of at least 10, 11, 12, 13, 14, 15, 16, 17, 18, 19, 20, 21, 22, 23, 24, 25, 26, 27, 28, 29 or 30 nucleotides in length and/or no more than 30, 35, 40, 45, 50, 55, 60, 65, 70, 75, 80, 85, 90, 95 or 100 nucleotides in length) on their surface that can serve binding site for an agent that comprises and/or is linked to second nucleic acid oligonucleotide (e.g., an oligonucleotide of at least 10, 11, 12, 13, 14, 15, 16, 17, 18, 19, 20, 21, 22, 23, 24, 25, 26, 27, 28, 29 or 30 nucleotides in length and/or no more than 30, 35, 40, 45, 50, 55, 60, 65, 70, 75, 80, 85, 90, 95 or 100 nucleotides in length) that specifically hybridizes to the first nucleic acid oligonucleotide. Methods for attaching oligonucleotides to the surface of bacterial cells are known in the art and described in, for example, Twite A. A., et al., Adv. Mater., 2012, 24(18):2380-5, which is hereby incorporated by reference. In some embodiments, the first oligonucleotide has a sequence that is at least 80%, 85%, 90%, 91%, 92%, 93%, 94%, 95%, 96%, 97%, 98%, 99% or 100% identical to a sequence of the second oligonucleotide. Exemplary methods for linking agents to oligonucleotides are provided, for example, in David A. Rusling & Keith R. Fox, Small Molecule-Oligonucleotide Conjugates, DNA Conjugates and Sensors, 2012, Ch3, 75-102, which is hereby incorporated by reference. In some embodiments, a cancer therapeutic is covalently linked to a single-stranded nucleic acid oligonucleotide that specifically hybridizes to a single-stranded nucleic acid oligonucleotide displayed on the cell surface of a bacteria described herein. The hybridized oligonucleotides hybridize and the resulting double-stranded nucleic acid duplex is stable for days. In some embodiments, the stability of the duplex is improved by incorporating phosphorothioate bonds (e.g., 1, 2, 3, 4, 5, 6, 7 or more phosphorothioate bonds) on the 5′ and/or 3′ ends of one or both oligonucleotides.

As used herein, the terms “polynucleotide”, and “nucleic acid” are used interchangeably and refer to a polymeric form of nucleotides, whether deoxyribonucleotides, ribonucleotides, or analogs thereof, in any combination and of any length. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides. A polynucleotide “specifically hybridizes” to a target sequence if the oligomer hybridizes to the target under physiological conditions, with a Tm substantially greater than 45° C., or at least 50° C., or at least 60° C.−80° C. or higher. Such hybridization corresponds to stringent hybridization conditions. At a given ionic strength and pH, the Tm is the temperature at which 50% of a target sequence hybridizes to a complementary polynucleotide. Such hybridization may occur with “near” or “substantial” complementarity of the oligonucleotide to the target sequence, as well as with exact complementarity.

In some embodiments, the bacteria described herein are linked to a cancer therapeutic through a biotin/streptavidin interaction. In some embodiments, the bacteria described herein are linked to biotin or to a cancer therapeutic using amine-reactive N-hydroxysuccinimide (NHS) esters or N-hydroxysulfosuccinimide (Sulfo-NHS) esters. NHS esters or Sulfo-NHS esters (Life Technologies can be made of virtually any carboxyl-containing molecule of interest by mixing the NHS or Sulfo-NHS with the carboxyl-containing molecule of interest and a dehydrating agent such as the carbodimide EDC using methods available in the art. Exemplary methods of labeling bacteria using NHS esters are provided in Bradburne J. A., et al., Appl. Environ. Microbiol., 1993, 59(3):663-8, which is hereby incorporated by reference.

In some embodiments, the bacteria described herein are linked to a cancer therapeutic through a sequence-specific DNA hybridization interaction. For example, a molecule of interest is covalently linked to a single-stranded DNA oligonucleotide and then attached to a bacterial cell that displays the complementary single-stranded DNA oligonucleotide on its cell surface. The two complementary oligonucleotides hybridize and the resulting double-stranded DNA duplex is stable for days. The stability of the DNA duplex and resistance to nucleases is further improved by incorporating 4 phosphorothioate bonds on the 5′ and 3′ ends of both oligonucleotides.

In some embodiments, unnatural amino acids containing ketones, azides, alkynes or other functional groups that are incorporated into surface-expressed proteins of the bacteria described herein are used to link the bacteria to a cancer therapeutic. Unnatural amino acids containing ketones, azides, alkynes or other functional groups known to one skilled in the art can be incorporated into target proteins in a residue-specific manner using, for example, an auxotrophic bacterial strain as described in Marquis H., et al., Infect. Immun., 1993, 61(9):3756-60, which is hereby incorporated by reference. For example, labeling of the bacterial cell surface can be accomplished by growing a methionine auxotrophic bacterial strain in the presence of the unnatural amino acid azidohomoalanine, which acts as a methionine surrogate and is incorporated during protein biosynthesis in place of methionine. Wild-type proteins on the bacterial surface that normally contain a surface-exposed methionine are now functionalized with a surface-exposed azide group, which can then modified with a molecule of interest that contains an alkyne group (e.g., an alkyne-derivatized small-molecule drug or an alkyne-derivatized protein) using Click Chemistry as described in Link A. J. & Tirrell D. A., Cell surface labeling of Escherichia coli via copper(I)-catalyzed [3+2] cycloaddition, J. Am. Chem. Soc., 2003, 125(37):11164-5, which is hereby incorporated by reference. After incorporation into the surface-expressed protein, these functional groups can serve as attachment points for a small-molecule of interest using, for example, the methods described in Prescher J. A. & Bertozzi C. R., Nat. Chem. Biol., 2005, 1(1):13-21, which is hereby incorporated by reference.

In some embodiments, the bacteria described herein is a gram-negative bacteria and the cancer therapeutic is linked to a surface-associated glycan. Linking a cancer therapeutic to a surface-associated glycan can be accomplished, for example, using a two-step metabolic/chemical labeling protocol. First, the surface-associated polymeric sugar is modified by metabolic labeling of the gram-negative bacterium with a chemically modified monosaccharide, which contains an azide functional group that is incorporated into the polymeric structure on the bacterial surface. Second, the cancer therapeutic is selectively ligated to the modified polymer on the bacterial cell surface using Click chemistry, for example, as described in Dumont A., et al., Angew. Chem. Int. Ed. Engl., 2012, 51(13):3143-6), which is hereby incorporated by reference.

In some embodiments, the bacteria described herein is a gram-positive bacteria and the cancer therapeutic is linked to the bacterial cell wall. The cell wall of gram-positive bacteria comprises of many interconnected layers of peptidoglycan (PG). A two-step metabolic/chemical labeling approach can be used for attaching an exogenously added molecule of interest to the PG. The gram-positive bacterial cells are first metabolically labeled by growing the cells in the presence of an alkyne-functionalized D alanine analog, which is incorporated into nascent PG layers during cell wall biosynthesis. Incorporation of the alkyne group then allows labeling of the PG with an azide-functionalized molecule of interest using the copper-catalyzed Click reaction as described in, for example, Siegrist M. S., et al., ACS Chem. Biol., 2013, 8(3):500-5, which is hereby incorporated by reference. In some embodiments, the gram-positive bacterial cells are grown in medium that contains a cyclooctyne-functionalized D alanine analog (e.g., exobcnDala or endobcnDala), which is then incorporated into the PG of the growing cells. The cells are washed with fresh medium and incubated with a cancer therapeutic that is derivatized with an azido-PEG₃ group to attach the molecule of interest to the PG in a copper-free reaction as described in, for example, Shieh P., et al., Proc. Natl. Acad. Sci. USA, 2014, 111(15):5456-61, which is hereby incorporated by reference. In some embodiments, the gram-positive bacterial cells are grown in medium that contains an unnatural D-amino acid with a norbornene (NB) group (e.g., D-Lys-NB-OH, D-Dap-NB-OH, D-Dap-NB-NH₂). The unnatural amino acid is metabolically incorporated into the PG of the growing bacterial cells and equips the bacterial cell surface with alkene functional groups with increased reactivity because of the strained alkene within the ring of the norbornene. The cells are then incubated with a tetrazine derivative of the cancer therapeutic to allow ligation of the cancer therapeutic to the PG, as described in Pidgeon S. E. & Pires M. M., Chem. Commun. (Camb)., 2015, 51(51):10330-3, which is hereby incorporated by reference.

In some embodiments, a cancer therapeutic is incorporated into the PG layer of a gram-negative bacterium described herein. Methods for incorporation molecules into the PG layer of a gram-negative bacterium are provided, for example, in Liechti G. W., et al., Nature, 2014, 506(7489):507-10, which is hereby incorporated by reference. In some embodiments, the gram-negative bacterium is grown in the presence of the D amino acid dipeptide EDA-DA (ethynyl-D alanine-D alanine) or DA-EDA (D alanine-ethynyl-D alanine). The EDA-DA, or DA-EDA, is incorporated into the PG layer of the actively growing bacteria and equips the PG with surface-exposed alkyne groups. Copper-catalyzed Click chemistry is used to attached a cancer therapeutic that contains a terminal azide group to the newly introduced alkyne groups of the PG layer. In some embodiments, a D amino acid derivative of a cancer therapeutic is be incorporated directly into the PG layer of a growing bacterium using, for example, the method described in Kuru E., et al., Nat. Protoc., 2015, 10(1):33-52, which is hereby incorporated by reference.

In some embodiments, the bacteria described herein are modified such that an immune modulatory protein, such as a cytokine is attached to the outside of a bacterium of interest using an attachment method described herein. Examples of immune modulating proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interlukin-1 alpha (“IL-1 alpha”), Interlukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophage inflammatory protein-1-delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (′BMP-5″), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“′TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MEC class I polypeptide-related sequence A (“MICA”), MEC class I polypeptide-related sequence B (“MICB”), NRG1-beta1, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”), Angiopoietin 1, Angiostatin, Catheprin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgp130, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor κ B (“RANK”). The immune modulatory protein can be made recombinantly using methods known to one skilled in the art. The immune modulatory protein can be presented on the surface of a bacterium using bacterial surface display, where the bacterium expresses a genetically engineered protein-protein fusion of e.g., a membrane protein and the immune modulatory protein.

In some embodiments, the bacteria described herein are engineered to express a peptide (e.g., an antigen) and/or a protein (e.g., a protein cancer therapeutic), intracellularly and/or on the bacterial surface (i.e., genetic surface display). For example, in some embodiments, the bacteria comprises a nucleic acid encoding a peptide or protein cancer therapeutic operably linked to transcriptional regulatory elements, such as a promotor. In some embodiments, the peptide or protein is constitutively expressed by the bacteria. In some embodiments, the peptide or protein is inducibly expressed by the bacteria (e.g., it is expressed upon exposure to a sugar or an environmental stimulus like low pH or an anaerobic environment). In some embodiments, the bacteria comprises a plurality of nucleic acid sequences that encode for multiple different recombinant peptides and/or proteins that can be expressed by the same bacterial cell.

In some embodiments, the bacteria displays a recombinantly produced peptide or protein (e.g., a peptide or protein cancer therapeutic) on its surface using a bacterial surface display system. Examples of bacterial surface display systems include outer membrane protein systems (e.g., LamB, FhuA, Ompl, OmpA, OmpC, OmpT, eCPX derived from OmpX, OprF, and PgsA), surface appendage systems (e.g., F pillin, FimH, FimA, FliC, and FliD), lipoprotein systems (e.g., INP, Lpp-OmpA, PAL, Tat-dependent, and TraT), and virulence factor-based systems (e.g., AIDA-1, EaeA, EstA, EspP, MSP1a, and invasin). Exemplary surface display systems are described, for example, in van Bloois, E., et al., Trends in Biotechnology, 2011, 29:79-86, which is hereby incorporated by reference.

In some embodiments, the bacteria display on their surface a peptide or protein of interest that alters the invasion or adhesion capability of the bacteria. In some embodiments, the protein that alters the invasion or adhesion capability of the bacteria is a bacterial adhesion, such as FadA (e.g., as described in Xu M., et al., J. Biol. Chem., 2007, 282(34):25000-9, which is hereby incorporated by reference) and or a synthetic adhesion (e.g., as described in Piñero-Lambea C., et al., ACS Synth. Biol., 2015, 4(4):463-73, which is hereby incorporated by reference). In some embodiments, the peptide or protein that alters the invasion or adhesion capability of the bacteria is an antibody or antigen binding fragment thereof having binding specificity for a cancer-specific antigen.

In some embodiments, the bacteria described herein comprise a cancer therapeutic (e.g., the cancer therapeutic is loaded into the bacteria prior to administration to a subject). In some embodiments, the cancer therapeutic is loaded into the bacteria by growing the bacteria in a medium that contains a high concentration (e.g., at least 1 mM) of the cancer therapeutic, which leads to either uptake of the cancer therapeutic during cell growth or binding of the cancer therapeutic to the outside of the bacteria. The cancer therapeutic can be taken up passively (e.g. by diffusion and/or partitioning into the lipophilic cell membrane) or actively through membrane channels or transporters. In some embodiments, drug loading is improved by adding additional substances to the growth medium that either increase uptake of the molecule of interest (e.g., Pluronic F-127) or prevent extrusion of the molecules after uptake by the bacterium (e.g., efflux pump inhibitors like Verapamil, Reserpine, Carsonic acid, or Piperine). In some embodiments, the bacteria is loaded with the cancer therapeutic by mixing the bacteria with the cancer therapeutic and then subjecting the mixture to electroporation, for example, as described in Sustarsic M., et al., Cell Biol., 2014, 142(1):113-24, which is hereby incorporated by reference. In some embodiments, the cells can also be treated with an efflux pump inhibitor (see above) after the electroporation to prevent extrusion of the loaded molecules.

### Cancer Therapeutic Agents

In certain aspects, the methods provided herein include the administration to a subject of a bacterium described herein either alone or in combination with another cancer therapeutic and/or an antibiotic. In certain embodiments, the bacteria are used to target a therapeutic agent to a tumor and/or a tissue. In certain aspects, the methods provided herein include the administration to a subject an antibiotic described herein either alone or in combination with another cancer therapeutic.

In some embodiments the bacterium is administered to the subject before the cancer therapeutic is administered (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments the bacterium is administered to the subject after the cancer therapeutic is administered (e.g., at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the bacterium and the cancer therapeutic are administered to the subject simultaneously or nearly simultaneously (e.g., administrations occur within an hour of each other). In some embodiments, the subject is administered an antibiotic before the bacterium is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before), In some embodiments, the subject is administered an antibiotic after the bacterium is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the bacterium and the antibiotic are administered to the subject simultaneously or nearly simultaneously (e.g., administrations occur within an hour of each other). In some embodiments, the bacterium is linked to, comprises and/or expresses the cancer therapeutic.

In some embodiments, the cancer therapeutic is a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegal 1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the cancer therapeutic is a cancer immunotherapy agent. Immunotherapy refers to a treatment that uses a subject's immune system to treat cancer, e.g., checkpoint inhibitors, cancer vaccines, cytokines, cell therapy, CAR-T cells, and dendritic cell therapy. Non-limiting examples of immunotherapies are checkpoint inhibitors include Nivolumab (BMS, anti-PD-1), Pembrolizumab (Merck, anti-PD-1), Ipilimumab (BMS, anti-CTLA-4), MEDI4736 (AstraZeneca, anti-PD-L1), and MPDL3280A (Roche, anti-PD-L1). Other immunotherapies may be tumor vaccines, such as Gardail, Cervarix, BCG, sipulencel-T, Gp100:209-217, AGS-003, DCVax-L, Algenpantucel-L, Tergenpantucel-L, TG4010, ProstAtak, Prostvac-V/R-TRICOM, Rindopepimul, E75 peptide acetate, IMA901, POL-103A, Belagenpumatucel-L, GSK1572932A, MDX-1279, GV1001, and Tecemotide. Immunotherapy may be administered via injection (e.g., intravenously, intratumorally, subtumorally, peritumorally, subcutaneously, or into lymph nodes), but may also be administered orally, topically, or via aerosol. Immunotherapies may comprise adjuvants such as cytokines.

In some embodiments, the immunotherapy agent is an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, atezolizumab, avelumab, durvalumab, ipilimumab, nivolumab, pembrolizumab, pidilizumab, AMP-224, AMP-514, BGB-A317, STI-A1110, TSR-042, RG-7446, BMS-936559, MEDI-4736, MSB-0020718C, AUR-012 or STI-A1010.

In certain embodiments, immune checkpoint inhibitors can be an inhibitory nucleic acid molecule (e.g., an siRNA molecule, an shRNA molecule or an antisense RNA molecule) that inhibits expression fo an immune checkpoint protein that inhibits expression of an immune checkpoint protein.

In some embodiments, the immune checkpoint inhibitor is a siRNA molecule. Such siRNA molecules should include a region of sufficient homology to the target region, and be of sufficient length in terms of nucleotides, such that the siRNA molecule down-regulate target RNA (e.g., RNA of an immune checkpoint protein). The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 29, 21, 22, 23, 24, or 25 nucleotide pairs, and one or more overhangs, such as one or two 3′ overhangs, of 2-3 nucleotides.

In some embodiments, the immune checkpoint inhibitor is a snRNA molecule. A “small hairpin RNA” or “short hairpin RNA” or “snRNA” includes a short RNA sequence that makes a tight hairpin turn that can be used to silence gene expression via RNA interference. The shRNAs provided herein may be chemically synthesized or transcribed from a transcriptional cassette in a DNA plasmid. The snRNA hairpin structure is cleaved by the cellular machinery into siRNA, which is then bound to the RNA-induced silencing complex (RISC).

In some embodiments, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40, 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some embodiments, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length), or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. patent application publication number 2011/0071208, the disclosure of which is herein incorporated by reference in its entirety for all purposes.

In some embodiments, the immune checkpoint inhibitor is an antisense oligonucleotide compounds that inhbits expression of an immune checkpoint protein. In certain embodiments, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain embodiments, antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

The inhibitory nucleic acid molecule can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

In the present methods, the inhibitory nucleic acid molecule can be administered to the subject, for example, as naked nucleic acid, in combination with a delivery reagent, and/or as a nucleic acid comprising sequences that express an interfering nucleic acid molecule. In some embodiments the nucleic acid comprising sequences that express the interfering nucleic acid molecules are delivered within vectors, e.g. plasmid, viral and bacterial vectors. Any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety. Exemplary interfering nucleic acid delivery systems are provided in U.S. Pat. Nos. 8,283,461, 8,313,772, 8,501,930. 8,426,554, 8,268,798 and 8,324,366, each of which is hereby incorporated by reference in its entirety.

In some embodiments, the immunotherapy agent is an antibody or antigen binding fragment thereof that, for example, binds to a cancer-associated antigen. Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGFS, FLT3-ITD, FN1, G250/MN/CAIX, GAGE-1,2,8, GAGE-3,4,5,6,7, GAS7, glypican-3, GnTV, gp100/Pme117, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUCSAC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAXS, PBF, pml-RARalpha fusion protein, polymorphic epithelial mucin (“PEM”), PPP1R3B, PRAME, PRDXS, PSA, PSMA, PTPRK, RAB38/NY-MEL-1, RAGE-1, RBAF600, RGSS, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRll, TPBG, TRAG-3, Triosephosphate isomerase, TRP-1/gp75, TRP-2, TRP2-INT2, tyrosinase, tyrosinase (“TYR”), VEGF, WT1, XAGE-1b/GAGED2a. In some embodiments, the antigen is a neo-antigen.

In some embodiments, the immunotherapy agent is a cancer vaccine and/or a component of a cancer vaccine (e.g., an antigenic peptide and/or protein). The cancer vaccine can be a protein vaccine, a nucleic acid vaccine or a combination thereof. For example, in some embodiments, the cancer vaccine comprises a polypeptide comprising an epitope of a cancer-associated antigen. In some embodiments, the cancer vaccine comprises a nucleic acid (e.g., DNA or RNA, such as mRNA) that encodes an epitope of a cancer-associated antigen. In some embodiments, the nucleic acid is a vector (e.g., a bacterial vector, viral vector). Examples of bacterial vectors include, but are not limited to, Mycobacterium bovis (BCG), Salmonella Typhimurium ssp., Salmonella Typhi ssp., Clostridium sp. spores, Escherichia coli Nissle 1917, Escherichia coli K-12/LLO, Listeria monocytogenes, and Shigella flexneri. Examples of viral vectors include, but are not limited to, vaccinia, adenovirus, RNA viruses, and replication-defective avipox, replication-defective fowlpox, replication-defective canarypox, replication-defective MVA and replication-defective adenovirus.

In some embodiments, the cancer immunotherapy comprises administration of an antigen presenting cell (APC) primed with a cancer-specific antigen. In some embodiments, the APC is a dendritic cell, a macrophage or a B cell.

Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-AL dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGFS, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-AL MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, MEL Melan-A/MART-1, Meloe, Midkine, MMP-2, MMP-7, MUC1, MUCSAC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAXS, PBF, pml-RARalpha fusion protein, polymorphic epithelial mucin (“PEM”), PPP1R3B, PRAME, PRDXS, PSA, PSMA, PTPRK, RAB38/NY-MEL-1, RAGE-1, RBAF600, RGSS, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRll, TPBG, TRAG-3, Triosephosphate isomerase, TRP-1/gp75, TRP-2, TRP2-INT2, tyrosinase, tyrosinase (“TYR”), VEGF, WT1, XAGE-1b/GAGED2a. In some embodiments, the antigen is a neo-antigen.

In some embodiments, the cancer immunotherapy comprises administration of a cancer-specific chimeric antigen receptor (CAR). In some embodiments, the CAR is administered on the surface of a T cell. In some embodiments, the CAR binds specifically to a cancer-associated antigen.

In some embodiments, the cancer immunotherapy comprises administration of a cancer-specific T cell to the subject. In some embodiments, the T cell is a CD4+ T cell. In some embodiments, the CD4+ T cell is a TH1 T cell, a TH2 T cell or a TH17 T cell. In some embodiments, the T cell expresses a T cell receptor specific for a cancer-associated antigen.

In some embodiments, the cancer vaccine is administered with an adjuvant. Examples of adjuvants include, but are not limited to, an immune modulatory protein, Adjuvant 65, α-GalCer, aluminum phosphate, aluminum hydroxide, calcium phosphate, β-Glucan Peptide, CpG DNA, GPI-0100, lipid A, lipopolysaccharide, Lipovant, Montanide, N-acetyl-muramyl-L-alanyl-D-isoglutamine, Pam3CSK4, quil A and trehalose dimycolate.

In some embodiments, the immunotherapy agent is an immune modulating protein to the subject. In some embodiments, the immune modulatory protein is a cytokine. Examples of immune modulating proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interlukin-1 alpha (“IL-1 alpha”), Interlukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophage inflammatory protein-1-delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MEC class I polypeptide-related sequence A (“MICA”), MEC class I polypeptide-related sequence B (“MICB”), NRG1-beta1, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”), Angiopoietin 1, Angiostatin, Catheprin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgp130, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor κ B (“RANK”).

In some embodiments, the cancer therapeutic is a radioactive moiety that comprises a radionuclide. Exemplary radionuclides include, but are not limited to Cr-51, Cs-131, Ce-134, Se-75, Ru-97, I-125, Eu-149, Os-189m, Sb-119, I-123, Ho-161, Sb-117, Ce-139, In-111, Rh-103m, Ga-67, T1-201, Pd-103, Au-195, Hg-197, Sr-87m, Pt-191, P-33, Er-169, Ru-103, Yb-169, Au-199, Sn-121, Tm-167, Yb-175, In-113m, Sn-113, Lu-177, Rh-105, Sn-117m, Cu-67, Sc-47, Pt-195m, Ce-141, I-131, Tb-161, As-77, Pt-197, Sm-153, Gd-159, Tm-173, Pr-143, Au-198, Tm-170, Re-186, Ag-111, Pd-109, Ga-73, Dy-165, Pm-149, Sn-123, Sr-89, Ho-166, P-32, Re-188, Pr-142, Ir-194, In-114m/In-114, and Y-90.

In some embodiments, the cancer therapeutic is a an angiogenesis inhibitor to the subject. Examples of such angiogenesis inhibitors include, but are not limited to Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (CometrigTM)

### Cancer

In some embodiments, the methods and compositions described herein relate to the treatment of cancer. In some embodiments, any cancer that forms a tumor can be treated using the methods described herein. Examples of cancers that may treated by methods and compositions described herein include, but are not limited to, cancer cells from the bladder, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; and granular cell tumor.

In some embodiments, the methods and compositions provided herein relate to the treatment of a carcinoma. The term “carcinoma” refers to a malignant growth made up of epithelial cells tending to infiltrate the surrounding tissues, and/or resist physiological and non-physiological cell death signals and gives rise to metastases. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

In some embodiments, the methods and compositions provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and compositions described herein include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

Particular categories of tumors that can be treated using methods and compositions described herein include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

Cancers treated in certain embodiments also include precancerous lesions, e.g., actinic keratosis (solar keratosis), moles (dysplastic nevi), acitinic chelitis (farmer's lip), cutaneous horns, Barrett's esophagus, atrophic gastritis, dyskeratosis congenita, sideropenic dysphagia, lichen planus, oral submucous fibrosis, actinic (solar) elastosis and cervical dysplasia.

Cancers treated in some embodimentsinclude non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

In some embodiments, the cancer is colon cancer.

### EXAMPLES

### Example 1

DNA was extracted from 18 paired biopsy samples (colon tumor and normal adjacent tissue) from 9 patients and amplified for 16S ribosomal DNA. The extracted DNA was profiled for 16S sequencing using MiSeq run generating 26,381,358 bacterial reads that were classified using a proprietary database of microbial genomes containing a total of 37,183 bacterial/fungal/archaeal/protozoa genomes. A total of 717 species and 235 genera were identified in the biopsy samples.

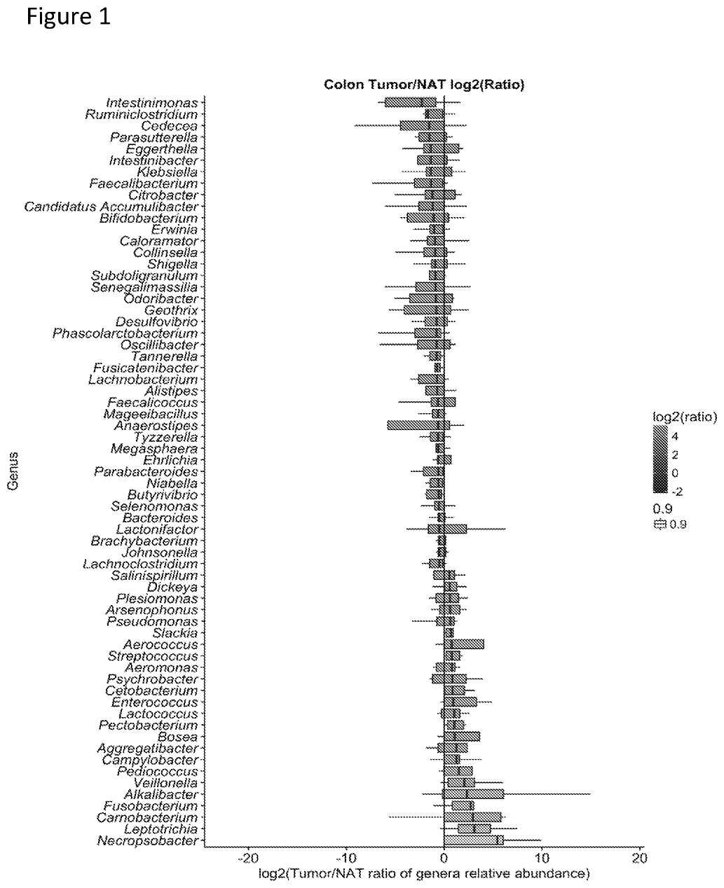

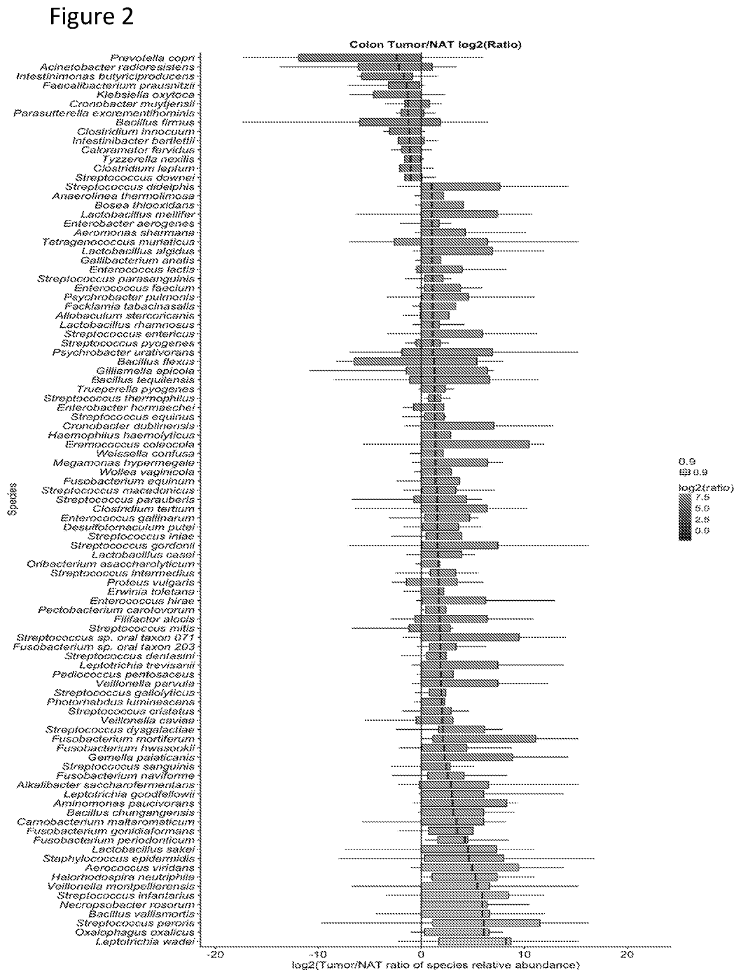

The analysis reflects the log 2 fold change ratio between the relative abundance of genera or species from tumor and the normal adjacent tissue from each subject. The median log 2 (tumor/normal adjacent tissue) ratios of relative abundance of the identified bacterial genera are provided in Tables 1 and 3, respectively. The median log 2 (tumor/normal adjacent tissue) ratios of relative abundance of the identified bacterial species are provided in Tables 2 and 4, respectively. The distribution of all samples and the median log 2(ratio) for each identified genus and species is presented in FIGS. 1 and 2, respectfully.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating a subject who has a tumor, the method comprising administering to the tumor a pharmaceutical composition comprising a bacterium of a genus listed in Table 1 or of a species listed in Table 2.

2. A method of augmenting a microbiome in a subject who has a tumor, the method comprising administering to the tumor a pharmaceutical composition comprising a bacterium of a genus listed in Table 1 or of a species listed in Table 2.

3. The method of claim 1, wherein the pharmaceutical composition is administered orally, intravenously, intratumorally, or subcutaneously.

4. The method of claim 1, wherein the pharmaceutical composition comprises bacteria of two or more genera listed in Table 1 or of two or more species listed in Table 2.

5. The method of claim 1, wherein the pharmaceutical composition does not comprise a bacterium of a genus listed in Table 3 or of a species listed in Table 4.

6. The method of claim 1, wherein at least 50% of the bacteria in the pharmaceutical composition are bacteria of a genus listed in Table 1 or of a species listed in Table 2.

7-14. (canceled)

15. The method of claim 1, wherein the pharmaceutical composition comprises at least 1×10⁶ colony forming units (CFUs) of bacteria in the pharmaceutical composition are bacteria of a genus listed in Table 1 or of a species listed in Table 2.

16-21. (canceled)

22. The method of claim 1, wherein the pharmaceutical composition is administered to the subject in two or more doses.

23. The method of claim 22, wherein the administration of the two or more doses are separated by at least 1 hour.

24-30. (canceled)

31. The method of claim 1, further comprising administering to the subject an antibiotic prior to the administration of the pharmaceutical composition.

32-38. (canceled)

39. The method of claim 1, wherein the pharmaceutical composition comprises live bacteria, attenuated bacteria, or killed bacteria.

40-43. (canceled)

44. A method of treating a subject who has a tumor, the method comprising depleting the tumor of bacteria of a genus listed in Table 3 or of a species listed in Table 4.

45. A method of changing a tumor microbiome in a subject who has a tumor, the method comprising depleting the tumor of bacteria of a genus listed in Table 3 or of a species listed in Table 4.

46-51. (canceled)

52. A method of delivering an agent to a subject with a tumor, the method comprising administering to the subject a pharmaceutical composition comprising bacteria linked to the agent, wherein the bacteria is of a genus listed in Table 3 or of a species listed in Table 4.

53-55. (canceled)

56. A method of delivering an agent to a subject with a tumor, the method comprising administering to the subject a pharmaceutical composition comprising bacteria that express the agent, wherein the bacteria is of a genus listed in Table 3 or of a species listed in Table 4.

57-61. (canceled)

62. A method of delivering an agent to a subject with tumor, the method comprising administering to the subject a pharmaceutical composition comprising bacteria comprising the agent, wherein the bacteria is of a genus listed in Table 3 or of a species listed in Table 4.

63-73. (canceled)

74. The method of claim 1, wherein the tumor is colon cancer tumor.

75. The method of claim 1, wherein the method further comprises administering to the subject a cancer therapy.

76. The method of claim 75, wherein the cancer therapy comprises the administration of a chemotherapy agent to the subject.

77. (canceled)

78. The method of claim 75, wherein the cancer therapy comprises cancer immunotherapy.

79. The method of claim 78, wherein the cancer immunotherapy comprises administering an immune checkpoint inhibitor to the subject.

80-81. (canceled)

82. The method of claim 79, wherein the immune checkpoint inhibitor is selected from the group consisting of atezolizumab, avelumab, durvalumab, ipilimumab, nivolumab, pembrolizumab, pidilizumab, AMP-224, AMP-514, BGB-A317, STI-A1110, TSR-042, RG-7446, BMS-936559, MEDI-4736, MSB-0020718C, AUR-012 or STI-A1010.

83-124. (canceled)

125. The method of claim 75, wherein the cancer therapy comprises administering an antibiotic to the subject.

126-127. (canceled)

128. The method of claim 1, wherein the method further comprises administering a prebiotic to the subject.

129-133. (canceled)
